(19) United States
(12) Patent Application Publication
(10) Pub. No.: US 2023/0372087 A1
(43) Pub. Date: Nov. 23, 2023

(54) ENGINEERING-DESIGN-BASED WORKFLOW FOR VALVE RECONSTRUCTION

(71) Applicant: Children's Medical Center Corporation, Boston, MA (US)

(72) Inventors: Peter E. Hammer, Needham, MA (US); David Hoganson, Chestnut Hill, MA (US)

(73) Assignee: Children's Medical Center Corporation, Boston, MA (US)

(21) Appl. No.: 18/030,473

(22) PCT Filed: Oct. 7, 2021
(86) PCT No.: PCT/US2021/053973

§ 371 Date: Apr. 5, 2023

Related U.S. Application Data
(60) Provisional application No. 63/088,663, filed on Oct. 7, 2020.

(51) Int. Cl. A61F 2/24

(52) U.S. Cl. CPC A61F 2/2415; A61F 2/2496; A61L 2430/20

(57) ABSTRACT

An engineering workflow for prospective design of heart valve leaflet grafts for surgical heart valve reconstruction is disclosed. The engineering workflow includes utilizing a quantitative description of one or more mechanical properties of one or more materials to be used for the heart valve reconstruction to prescribe a size and/or a shape of the materials to achieve a predetermined configuration of a final reconstructed heart valve in its physiological working state for a given patient.

### CROSS REFERENCE TO RELATED APPLICATIONS

This application claims the benefit under 35 U.S.C. § 119(e) of U.S. Provisional Application Ser. No. 63/088,663, titled “Engineering-Design-Based Workflow for Aortic Valve Reconstruction,” filed on Oct. 7, 2020, which is incorporated herein by reference in its entirety.

### FIELD

Disclosed embodiments relate to an engineering workflow for preparing a material to be implanted into a subject, such as heart valve leaflet grafts for surgical heart valve reconstruction.

### BACKGROUND

The normal human heart contains four valves, which control the direction of blood flow through the heart. The valves open and close passively due to pressure gradients generated as the muscular walls of the heart chambers contract and relax. The aortic valve is positioned at the junction of the left ventricle and the aorta. It is comprised of three thin flaps, referred to as leaflets, that open to allow blood ejected from the left ventricle to enter the systemic arteries then close as the left ventricle relaxes to maintain pressure in the aorta while the left ventricle refills. In a healthy valve, the closed leaflets come together with some degree of overlap or redundancy. This area of overlap of adjacent, closed leaflets is called the area of coaptation.

Valve disease refers to conditions in which a valve fails either to open adequately, close adequately, or both. Severe valve disease can be treated either by replacing the valve with a prosthetic valve or by surgically altering the valve. The decision whether to replace or surgically repair a valve is multifactorial, but valve repair, including reconstructing one or more of the leaflets using some type of non-growing patch material, is often the treatment of choice in children as well as some subsets of adult patients.

The conventional approach for heart valve reconstruction is subjective and based on the experience and preferences of the individual surgeon. According to this conventional approach, a single piece or multiple pieces of material are cut free hand from a larger piece of autograft, homograft, xenograft, or synthetic material in the operating room during surgery according to individualized methods. The surgeon will often make one or more measurements of patient cardiovascular anatomy to help guide sizing of the piece or pieces.

### BRIEF SUMMARY

According to one aspect, a method of preparing an implantable material includes: obtaining a target configuration for a biological valve; obtaining one or more characteristics of the biological valve to be reconstructed; obtaining one or more mechanical characteristics of the implantable material; and determining, based at least in part on the target configuration, the one or more biological valve characteristics, and the one or more mechanical characteristics of the implantable material, a pattern for the implantable material configured to reconstruct the biological valve.

According to another aspect, a non-transitory computer readable storage media comprising processor executable instructions that when executed perform a method for preparing an implantable material comprising the steps of: obtaining a target configuration for a biological valve; obtaining one or more characteristics of the biological valve to be reconstructed; obtaining one or more mechanical characteristics of the implantable material; and determining, based at least in part on the target configuration, the one or more biological valve characteristics, and the one or more mechanical characteristics of the implantable material, a pattern for the implantable material configured to reconstruct the biological valve.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF DRAWINGS

Non-limiting embodiments of the present disclosure will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1A is a top view of an aortic valve in which the aorta has been cut above, or distal to, the level of the valve;

FIG. 1B is a side view showing a section of the aortic valve cut at the position of cutline 1B-1B shown in FIG. 1A;

FIG. 2 depicts an engineering workflow according to one illustrative embodiment;

FIG. 3 is a flowchart illustrating the steps of an engineering workflow according to one illustrative embodiment; and

FIG. 4 is a top view of a patterned implantable material configured to repair a defective aortic valve according to one illustrative embodiment.

### DETAILED DESCRIPTION

Generally speaking, to treat a defect in a biological valve (e.g., a heart valve) in a patient, a clinician may elect to surgically repair such a defective heart valve by implanting a material into the patient at the site of the defect. To implant such a material, the clinician may wish to prepare the material by cutting an appropriate pattern or patterns for sizing and shaping the material prior to surgical implantation. However, primary challenges associated with conventional implantable materials include determining the shape and/or size of the implantable material or portions thereof. Particularly, a clinician may need to estimate the three-dimensional properties (e.g., as exhibited after implantation into a patient) of a two-dimensional implantable material. In particular, after implantation, the implantable material may stretch between an intraoperative, zero-pressure state and one or more physiological conditions (e.g., pressurized states). Accordingly, it may be desirable for the size and/or shape of the material to account for such stretch.

Conventionally, a clinician may apply a qualitative approach to selecting and preparing an implantable material for reconstructing a biological valve. For example, the clinician may inspect the biological valve before or during surgery and qualitatively select an implantable repair material (e.g., off the shelf). Such an approach may result in a variable fit, which may result in premature wear or failure of the repair material. Accordingly, the inventors have recognized the advantages of a quantitative, tailored workflow for preparing an implantable material for reconstructing a biological valve for an individual patient.

In addition, it also may be desirable for the clinician to account for how much a material may stretch between a state in which the material is prepared and a state under which a valve, reconstructed from the material, may be configured to function (e.g., where hemodynamic forces and pressures may stretch the material, in some cases to 40 percent or more relative to its resting unstretched condition). Additionally, appropriate and/or widely used implantable materials may exhibit some degree of anisotropy (e.g., the material may exhibit different stress strain characteristics in different directions of deformation). Some conventionally used valve reconstruction materials may be highly anisotropic, stretching up to four times as much in a direction of minimum stiffness as compared to a direction of maximum stiffness off the material.

The inventors have also recognized that valve reconstruction in a child may present additional challenges, including a desire to reconstruct the valve such that the valve may retain its function for as long as possible (e.g., as the child grows), thus sparing the patient additional surgeries during childhood.

Conventionally, implantable materials are prepared subjectively (e.g., according to the qualitative training and/or experience of a clinician). For example, when surgically reconstructing a biological valve, a clinician may consider many variables that may affect function of the reconstructed valve following surgery. These may include: three-dimensional spatial details of the patient's anatomy; the mechanical properties of the native valve and its surroundings (e.g., an aortic root); the mechanical properties of the material chosen to reconstruct the valve; the size, shape, and/or orientation of the piece or pieces of material used to reconstruct the valve; the method for surgically implanting the piece or pieces of material; and the physiological variables relating to the conditions under which the valve may function following a surgical repair. The clinician may normally attempt to integrate all these complex variables in the operating room without the aid of quantitative methods or tools, instead determining the material choice as well as the size and shape of the piece or pieces mainly based on experience and intuition. Accordingly, conventional surgical valve reconstruction results may vary greatly based on the experience of an individual clinician.

In an effort to standardize methods for biological valve reconstruction, products have been developed and commercialized that utilize a set of devices to determine the size of a patient's valve together with a corresponding set of templates for tracing and/or cutting pieces of material, from which the valve may be reconstructed. However significant limitations with such products remain. For example, the material used for valve reconstruction may vary among patients depending on factors such as the availability of autologous tissues for harvest and the preference of the clinician. The conventional approaches to standardization may not account for the material mechanical properties of both native structures (e.g., the valve to be repaired and support tissue) and specific valve reconstruction materials when cut to the prescribed sizes and shapes of the pieces of material from which the valve may be reconstructed.

Furthermore, the current approaches to standardization may not account for material anisotropy, that is, the presence of a direction along the material in which the deformation of the material is a minimum under a given strain with respect to all other directions in the material. Thus, these current approaches may not be capable of predicting the sizes and/or shapes for the piece or pieces of material to be used to reconstruct a valve in a given patient, particularly with respect to withstanding and functioning under the various physiological states that may be present within a patient. Moreover, conventional products and/or methods may not be capable of accounting for or accommodating changing physiological demands on the valve, such as those imposed by patient growth.

In view of the above, the inventors have recognized the advantages of a quantitative method for specifying the shape and/or size of a piece or multiple pieces of material to be used to reconstruct a heart valve, such that the heart valve reconstructed from this piece, or pieces, of material is capable of achieving target open and closed configurations as well as accounting for the mechanical properties of the reconstruction material(s). Furthermore, it is desirable in growing patients (e.g., children) that a method for preparing a piece, or pieces, of material for valve reconstruction produce a reconstructed valve that accommodates patient growth.

Accordingly, the workflow disclosed herein relates to a fully quantitative approach to the surgical reconstruction of a biological valve (e.g., the aortic valve of the heart). When surgically reconstructing a biological valve, a clinician may consider many variables that may affect function of the reconstructed valve following surgery. As described herein these may include: three-dimensional spatial details of the patient's anatomy; the mechanical properties of the native valve and its surroundings (e.g., an aortic root); the mechanical properties of the material chosen to reconstruct the valve; the size, shape, and/or orientation of the piece or pieces of material used to reconstruct the valve; the method for surgically implanting the piece or pieces of material; and the physiological variables relating to the conditions under which the valve may function following a surgical repair. The variables, including those listed above, may be quantifiable and may be directly measured or estimated prior to or during surgery. Furthermore, the variables are related to the postoperative function of the valve, for example according to physical laws of mechanics. The workflow described herein may provide a clinician with a quantitatively determined pattern or patterns for use in cutting a piece or pieces of material for use in a biological valve reconstruction such that patient-specific features and dimensions of the valve are achieved postoperatively when the valve is operating under physiological conditions. As will be appreciated by one of skill in the art, in some embodiments, the pattern may be configured to reconstruct the valve such that a desired coaptation height and/or area is achieved between two or more leaflets of the valve.

According to one embodiment, an engineering workflow method may include utilizing a quantitative description of the mechanical properties of the material to be used for the reconstructed valve in order to determine a size and/or a shape of the reconstruction materials such that the reconstruction materials are configured to repair a biological valve such that the valve is appropriately configured to operate in desired working state in a given patient following implantation.

For example, in some embodiments, a method of preparing an implantable material (e.g., for a clinician to use when repairing a biological valve) is disclosed. The method may include obtaining a target configuration for the implantable material. The target configuration may include a target size, a target shape, a target maximum and/or minimum elasticity, target deformation characteristics, coaptation height and/or area (e.g., an overlapping height and/or area between two or more leaflets that make up a reconstructed valve), curvature, angle relative to the surroundings, tissue strength, and/or any other suitable metric. The method may further include obtaining one or more characteristics (e.g., mechanical characteristics) of a biological valve to be reconstructed. The one or more characteristics of the biological valve may include a size, a shape, an elasticity profile, a stress-strain relationship of the biological valve, coaptation height and/or area (e.g., an overlapping height and/or area between two or more leaflets that make up a reconstructed valve), curvature, angle relative to the surroundings, tissue strength, and/or any other suitable characteristic. Relatedly, characteristics (e.g., mechanical characteristics) of an implantable material may be obtained such as a size, a shape, an elasticity profile, a stress-strain relationship of the implantable material, coaptation height and/or area (e.g., an overlapping height and/or area between two or more leaflets that make up a reconstructed valve), curvature, angle relative to the surroundings, tissue strength, and/or any other suitable characteristic. In turn, a pattern (e.g., for cutting the implantable material) may be determined based at least in part on the target configuration of the implantable material, the characteristics of the biological valve, and the characteristics of the implantable material. In some embodiments, this method may be embodied by a non-transitory computer readable storage media that includes processor executable instructions that when executed implement the methods disclosed herein.

As will be appreciated by one of skill in the art, the characteristics (e.g., the size, shape, elasticity profile, and/or stress-strain relationship) of the biological valve and/or the implantable material may be obtained in any suitable manner. For example, in some instances, the characteristics may be measured using mechanical tools (e.g., calipers, rulers, tape measures, etc.). In other instances, the characteristics may be electronically obtained. For example, a computer model of the relevant structures (e.g., the biological valve or the implantable material) may be generated such that the relevant measurements may be obtained. While the computer model would generally be structed as a three-dimensional model, embodiments including a two-dimensional computer model are also contemplated. Of course, the characteristics may be obtained in other suitable manners, depending on the application, as the disclosure is not so limited in this regard.

As will be appreciated by one of skill in the art, the stress-strain relationship of the biological valve and/or the implantable material may be obtained in any suitable manner. For example, in some embodiments, the stress-strain relationship of a biological valve and/or an implantable material includes relating a force per unit cross-section of the biological valve and/or the implantable material to a measure of an average deformation normalized to a size of the biological valve and/or the implantable material respectively.

Alternatively or in addition, the stress-strain relationship of the biological valve and/or the implantable material may be obtained in various regions of the biological valve and/or the implantable material respectively. For example, in some embodiments, the biological valve and/or the implantable material may exhibit anisotropic material properties (e.g., the biological valve and/or the implantable material has a direction in which the respective stress-strain relationship exhibits a maximal in-plane stiffness compared to all other directions). Accordingly, in some embodiments, the stress-strain relationship of the biological valve and/or the implantable material may be obtained in two or more different directions (e.g., axes). As will be appreciated by one of skill in the art, the stress-strain relationship of the biological valve and/or the implantable material may be measured using manual qualitative and quantitative measurements. In some embodiments, the stress-strain relationship of the biological valve and/or the implantable material may be measured using either contact or non-contact measurement methods known in the art. Of course, the stress-strain relationship of the biological valve and/or the implantable material may be measured in any suitable manner, depending on the application, as the disclosure is not so limited in this regard.

In some embodiments, one or more characteristics of the biological valve and/or the implantable material may be measured prior to surgery and/or at the time of surgery.

Once the pattern is determined, the pattern may be used as a template to cut or otherwise shape the implantable material prior to or during surgery. The implantable material may be cut in any suitable manner. For example, in some embodiments, the material may be cut by one or more of a scissor, a knife, a scalpel, a laser cutter, electrochemical cutting, die cutting, and/or any other suitable tool. In some specific embodiments, the implantable material may be laser cut to the determined pattern. In some embodiments, the pattern may be projected (e.g., using light or another suitable medium) onto the implantable material by a projector or other appropriate display to indicate how the material should be cut. Of course, the implantable material may be cut in other suitable manner including both automated and/or manual processes as the disclosure is not so limited in this regard.

As described herein, the pattern for the implantable material may be configured to function in various and/or changing physiological states within a given patient. For example, in the context of a heart valve, physiological state may include atrial systole, atrial diastole, ventricular systole, ventricular diastole, and/or any other suitable physiological condition. Further, in some embodiments, the pattern may be configured such that the reconstructed valve remains functional under both high stress (e.g., high heart rate and/or blood pressure) and low stress (e.g., low heart rate and/or blood pressure). Alternatively or in addition, the physiological conditions may include expected patient growth (e.g., stretching of the valve and/or the surroundings of the valve as the patient grows and/or ages). Of course, functionality of the heart valve under other physiological conditions is also contemplated, depending on the application, as the disclosure is not so limited in this regard.

The workflow described herein includes specifications for features of the reconstructed valve operating under physiological conditions. For example, in some embodiments, a functional valve may serve to selectively separate a higher-pressure biological region from a lower pressure biological region. A functional valve may be capable of taking on a closed configuration and an open configuration. The functional valve may separate two or more regions in the closed configuration, while the valve may allow for open fluid communication between the two or more regions in the open configuration. In some embodiments, the functional valve is passive, opening and closing in response to external pressures. For example, the valve may open under the pressure differential created when an adjacent heart chamber (e.g., the left ventricle in the context of an aorta), contracts. Relatedly, in some embodiments, when the adjacent heart chamber relaxes, the backpressure between the regions may serve to close the functional valve. Furthermore, a functional valve may be capable of opening rapidly so as to prevent loss of pressure and/or flow between the regions. In some embodiments, a functional valve may not obstruct other biological structures (e.g., a coronary ostia) during opening and/or closing.

In some embodiments, it may be desirable for the implantable material used in the reconstructed valve to be configured to accommodate for patient growth. For example, in some instances, a pediatric patient may have a biological valve reconstructed using an implantable material capable of functioning within the patient until the patient is fully grown or some other appropriate time period. Thus, in such embodiments, the implantable material used for the valve reconstruction may be oversized (e.g., while maintaining its functionality as described herein) to accommodate for patient growth.

In some embodiments, a method may include obtaining (e.g., measuring) the characteristics of the biological valve to be reconstructed and/or the surrounding tissue of the biological valve in the various physiological conditions. Particularly, in some embodiments, the target configuration for the implantable material (e.g., as described herein), may be based at least in part on the obtained characteristics of the biological valve to be reconstructed and/or the surroundings thereof in the various physiological conditions. Accordingly, it may be desirable to measure the characteristics of the biological valve and/or the surroundings thereof in a resting (e.g., unstressed or unpressurized) state. Thus, the resting state of the biological valve and/or the surroundings thereof may be compared to a resting (e.g., unstressed or unpressurized) state of the implantable material, for example, to determine a suitable target configuration, as described herein. Of course, the characteristics of the biological valve and/or the surroundings thereof may be obtained under any suitable physiological condition, depending on the application, as the disclosure is not so limited in this regard.

As will be appreciated by one of skill in the art, the characteristics of the biological valve to be reconstructed and/or the surrounding tissue of the biological valve in the various physiological conditions may be measured in any suitable manner. In some embodiments, the characteristics of the biological valve to be reconstructed and/or the surrounding tissue of the biological valve may be measured using magnetic resonance imaging, computed tomography, in vivo imaging, ultrasound, three-dimensional scanner, light detection and ranging systems, other manners of generating a three-dimensional image, mechanical testing and/or any other appropriate characterization method. Of course, the characteristics of the biological valve to be reconstructed and/or the surrounding tissue of the biological valve in the various physiological conditions may be measured in other suitable manners, depending on the application, as the disclosure is not limited in this regard.

Turning to the figures, specific non-limiting embodiments are described in further detail. It should be understood that the various systems, components, features, and methods described relative to these embodiments may be used either individually and/or in any desired combination as the disclosure is not limited to only the specific embodiments described herein.

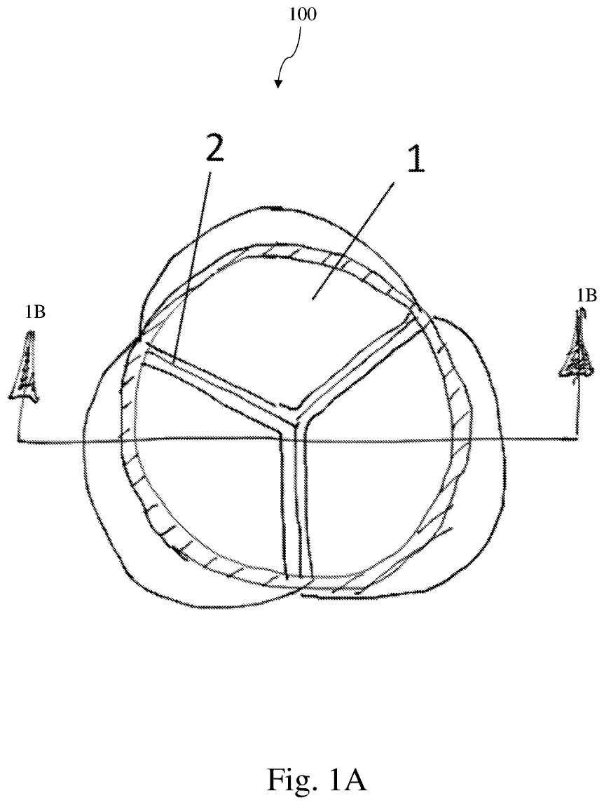

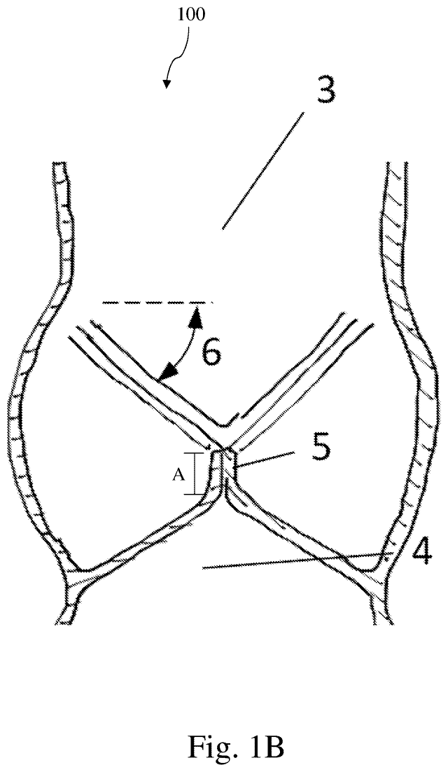

FIG. 1A illustrates a normal (e.g., non-defective) aortic valve 100 according to the prior art. When the normal aortic valve 100 is closed and pressurized at physiological levels of diastolic pressure, such a normally-functioning valve may exhibit one or more leaflets 1 that are arranged to come together, or coapt, with adjacent leaflets to form an unbroken seal 2. Seal 2 may be selectively openable under any suitable condition or conditions, including those described herein.

FIG. 1B illustrates a side view of the aortic valve 100 showing a section of the aortic valve 100 cut at the position of cutline 1B-1B shown in FIG. 1A. As shown in FIG. 1B, the closed valve 100 separates a region of higher pressure in the aortic root 3 from a region of lower pressure in the left ventricle 4. A well-functioning valve may also exhibit a minimum coaptation height A near overlap point 5, which is the distance over which adjacent leaflets overlap. Alternatively or additionally, a well-functioning valve may exhibit closed leaflets whose free edges form a downward angle 6, referred to herein as the free edge angle, from their point of attachment to the aortic root 3 toward a left ventricle of a heart of a patient.

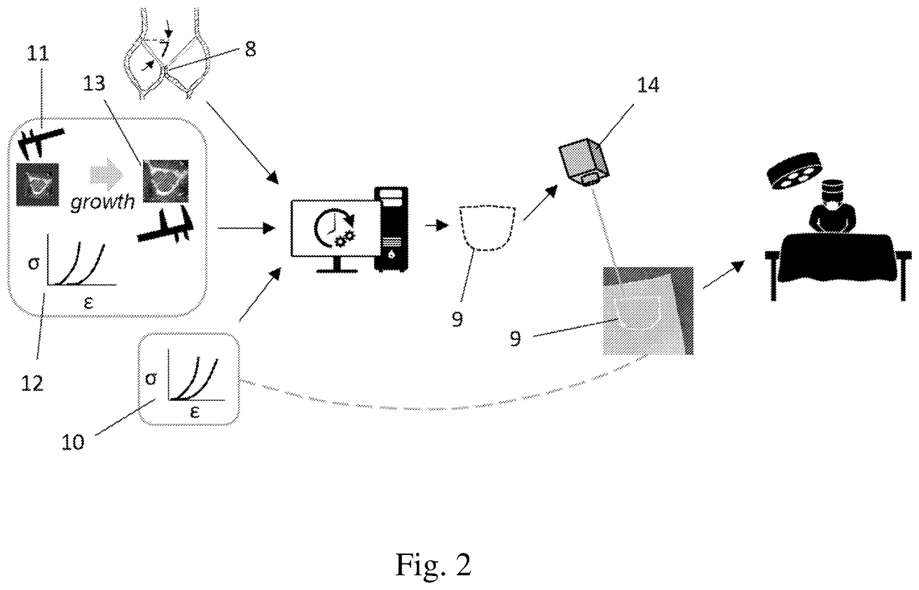

Referring to FIG. 2, target specifications for the reconstructed valve under physiological loading conditions may be based at least in part on the free edge angle 7 and a height of inter-leaflet coaptation (e.g., overlap) 8. Goals of the valve reconstruction workflow described herein include computation of a shape 9 into which an implantable material in its resting, unstressed state may be cut (e.g., in the operating room) in order to achieve a target closed valve configuration under the physiological conditions of a particular patient (e.g., those described herein). To determine the pattern, a change in the dimensions of the leaflets of the reconstructed valve may be calculated from a pressurized state, from which the valve target dimensions are obtained relative to the unpressurized, or resting, state from which the implantable material must be cut. For example, in some embodiments, a finite element method may be employed to obtain the valve target dimensions, which may be based on a numerical approximation of the equations of continuum mechanics. Alternatively or in addition, models of the mechanics of the solid structures using networks of masses and springs may be employed. Of course, the valve target dimensions may be obtained in any suitable manner, depending on the application, as the disclosure is not so limited in this regard.

Such exemplary methods for obtaining structural deformation data may be applied to a model of the valve in a resting, or unstressed, state in order to obtain stresses and strains throughout the valve in response to applied loads and/or deformations. In some embodiments of the workflow described herein, the goal is to compute a resting configuration of a valve structure after loads are removed, for example by applying an inverse finite element method. Discrete mass-spring methods may also be applied using this inverse modeling approach, though other approaches are also contemplated, as the disclosure is not so limited in this regard.

Referring again to FIG. 2, the disclosed methods of obtaining a resting configuration of a valve structure may be based on a quantitative description of mechanical properties 10 of an implantable material to be used to reconstruct the valve. Such material mechanical properties may be expressed in the form of a constitutive equation that relates stress, or force per unit cross-section of material, to strain, a measure of deformation normalized to the size of the structure (e.g., average deformation in a given direction or axis). This stress-strain relationship may be determined experimentally using established procedures for mechanical testing. In some instances, for the workflow proposed herein, the stress-strain relationships for implantable materials used to reconstruct the valve may be taken from published data or from experiments carried out prior to the surgery. Alternatively or in addition, the stress-strain relationship for the implantable materials used to reconstruct the valve may be estimated at the time of surgery.

The stress-strain relationships of implantable materials used to reconstruct heart valves are, in general, anisotropic, meaning that there exists a direction in the material in which the stress-strain relationship exhibits maximal in-plane stiffness compared to all other material directions. This direction of maximal in-plane stiffness is referred to as the principal material direction. Thus, changing an orientation of the principal material direction in a material or material piece that is implanted in the valve root may change a direction, relative to the implantation site, in which the material deforms the least. The workflow proposed herein includes specifying a principal material direction of the material used for reconstruction relative to a reference direction in the native structures (e.g., the native biological valve) at the implantation site. Alternatively or in addition, the principal material direction of the implantable material or piece or pieces thereof used for valve reconstruction may be configured such that an orientation of the principal material direction that results in a predicted valve function can be determined.

Referring again to FIG. 2, the methods described herein for obtaining the resting configuration of the valve may include a quantitative description of the size and shape of the surrounding of a native valve (e.g., an aortic root) of a given patient into which the implantable material or piece or pieces thereof are to be implanted. This quantitative description may include a set of discrete measurements of features of, for example, an aortic root 11 of the given patient. Alternatively or in addition, the measurement may take the form of a three-dimensional model of the anatomy of the aortic root 11 of the patient. The methods described herein for obtaining the resting configuration of the valve also may include a quantitative description 12 of mechanical properties of an aortic root 11 of the given patient. As for the stress-strain relationship for the implantable material used to reconstruct the valve, the stress-strain relationship for the aortic root 11 of the patient may be taken from published data or from experiments carried out prior to the surgery, may be estimated at the time of surgery, or obtained in any other suitable manner, as the disclosure is not so limited in this regard.

As described herein, the size and shape of the implantable material, or piece or pieces thereof, used to reconstruct the valve may be calculated based on multiple inputs. Such inputs include: a set of quantitative features describing a target configuration or multiple target configurations of the valve under physiological conditions; a set of discrete measurements of features of the surroundings of a patient (e.g., an aortic root of the patient); a stress-strain relationship of the surroundings of a patient (e.g., an aortic root of the patient); a stress-strain relationship of the implantable or piece or pieces thereof used to reconstruct the valve, and/or other appropriate parameters. In some embodiments, an output of the workflow includes a pattern or patterns for the implantable material or piece or pieces thereof. Alternatively or in addition, a further output may include an angular orientation of the pattern or patterns with respect to a principal material direction of the implantable material (e.g., in the case of an anisotropic implantable material).

For valve repair in children, the durability of a conventionally surgically reconstructed valve may be limited due to patient growth. Specifically, as a native aortic root of a pediatric patient grows, the reconstructed valve leaflets that are attached to it may not grow. Accordingly, in some instances, the reconstructed leaflets may be progressively pulled away from a center of the valve root and from one another. Such pulling away may continue as the patient grows until the leaflets of the reconstructed valve no longer appropriately to coapt, which may result in an incompetent or leaky valve.

Referring again to FIG. 2, in the case of the growing child, the workflow described herein may be applied such that a target configuration of the reconstructed valve is associated with a configuration of the valve at a size corresponding to a predicted size of the patient following a target degree of patient growth 13. The illustrative computational modeling methods described herein may be used to compute a valve configuration in the unstressed state relative to the predicted size of the patient following a target degree of patient growth 13.

In determining a pattern for the implantable material or the piece or pieces thereof, in the unstressed state, it may be desirable to transform the configuration of the valve leaflets in the unstressed state that is computed by the inverse computational modeling method described herein into a planar shape that may be traced or otherwise applied as a pattern on the material to be used to reconstruct the valve. This step, referred to herein as flattening, may be accomplished using illustrative computational mechanics approaches, such as the finite element method described herein, which may accounts for material mechanical properties in the flattening process. Alternatively or in addition, computer graphics approaches may be employed, which may include methods that approximate and/or neglect material mechanical properties.

In some embodiments, determination of the pattern may be obtained by searching a parameter space of all possible shapes, sizes, and/or orientations. The parameter space may be configured to account for desired values of coaptation and/or degree of patient growth.

The pattern or patterns for the piece or pieces of material used for valve reconstruction may be transferred to the material using any suitable method. Referring again to FIG. 2, a laser 14 or other light-based projector may be used to project the pattern or patterns 9 directly onto the implantable material. Alternatively or in addition, the pattern or patterns may be used to fabricate physical templates that may be used with a sterile marker or other method of transferring the pattern onto the implantable material. In either case, after the pattern is projected onto the implantable material, the implantable material may be cut to provide the desired patterned implantable material. In the case of an implantable material with anisotropic mechanical properties, the pattern for each piece may be aligned with respect to a principal material direction of the material from which the piece or pieces are to be cut.

An additional output of the workflow described herein may include instructions to be used to implant the implantable material into the patient. In the case where the piece or pieces are to be implanted with sutures, a workflow output may include a specification of the pattern of where sutures are to be placed on the implantable material and where corresponding sutures may be placed on the patient. In some instances, sutures may progress at a different rate on one piece of implantable material than on a different piece of implantable material and/or within the patient (e.g., in order to induce a predictable three-dimensional shape of the reconstructed valve or in order to incorporate redundancy of one material in the suture line with respect to the other).

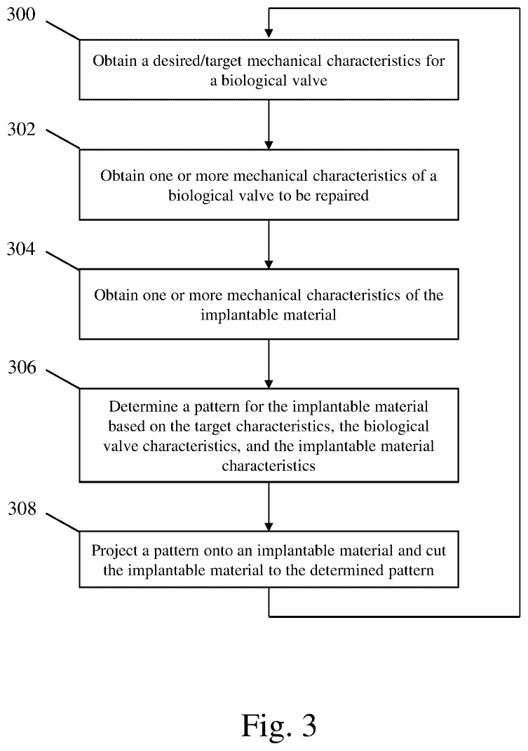

The workflow disclosed herein may be embodied as a method. FIG. 3 illustrates one such exemplary method. At step 300, target mechanical characteristics for a biological valve are obtained. At step 302, current mechanical characteristics for the biological valve are obtained (e.g., in a defective state). At step 304, one or more mechanical characteristics of an implantable material (e.g., a material to be used to repair the biological valve) are obtained. At step 306 a pattern for the implantable material is determined based at least in part on the obtained target characteristics, the biological valve characteristics, and the implantable material characteristics. At step 308, the implantable material is then cut according to the determined pattern. In some instances, this may include projecting a pattern onto the implantable material as described above.

The methods described herein may be executed in any suitable manner. For example, in some instances, the method may be executed using one or more processors configured to carry out the steps described herein. Alternatively or in addition, the method may be embodied as processor executable instructions stored in a non-transitory computer readable media.

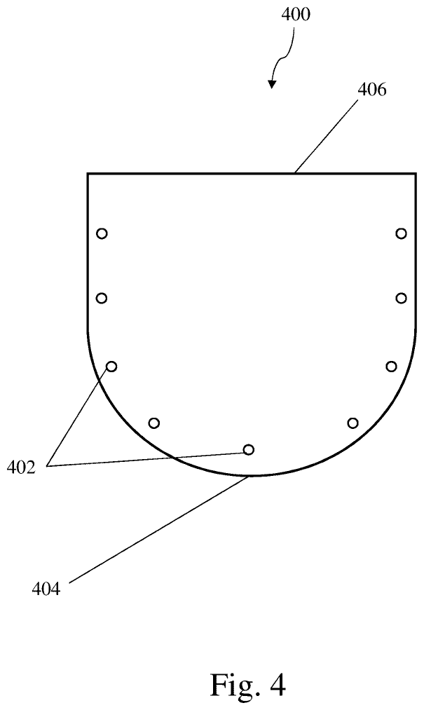

As described herein, the disclosed workflow may be used to produce a quantitatively tailored piece of an implantable material 400 for reconstruction of a biological valve. FIG. 4 illustrates one such example. In particular, a first end 404 of the piece of implantable material 400 may be tailored to sufficiently overlap with other portions of the associated biological valve as described herein. Moreover, a second end 406 of the piece of implantable material 400 opposite the first end 404 may be tailored to fit at an appropriate angle relative to the valve and the surrounding tissue as described herein. Further, as described herein the piece of implantable material 400 may include indicator marks 402, which provide a pattern for where a clinician may suture the piece of implantable material within the patient.

The above-described embodiments of the technology described herein can be implemented in any of numerous ways. For example, the embodiments may be implemented using hardware, software or a combination thereof. When implemented in software, the software code can be executed on any suitable processor or collection of processors, whether provided in a single computing device or distributed among multiple computing devices. Such processors may be implemented as integrated circuits, with one or more processors in an integrated circuit component, including commercially available integrated circuit components known in the art by names such as CPU chips, GPU chips, microprocessor, microcontroller, or co-processor. Alternatively, a processor may be implemented in custom circuitry, such as an ASIC, or semicustom circuitry resulting from configuring a programmable logic device. As yet a further alternative, a processor may be a portion of a larger circuit or semiconductor device, whether commercially available, semi-custom or custom. As a specific example, some commercially available microprocessors have multiple cores such that one or a subset of those cores may constitute a processor. Though, a processor may be implemented using circuitry in any suitable format.

Also, the processor may have one or more input and output devices. These devices can be used, among other things, to present a user interface. Examples of output devices that can be used to provide a user interface include display screens for visual presentation of output and speakers or other sound generating devices for audible presentation of output. Examples of input devices that can be used for a user interface include keyboards, individual buttons, and pointing devices, such as mice, touch pads, and digitizing tablets. As another example, a computing device may receive input information through speech recognition or in other audible format.

Such processors may be interconnected by one or more networks in any suitable form, including as a local area network or a wide area network, such as an enterprise network or the Internet. Such networks may be based on any suitable technology and may operate according to any suitable protocol and may include wireless networks, wired networks or fiber optic networks.

Also, the various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, the embodiments described herein may be embodied as a computer readable storage medium (or multiple computer readable media) (e.g., a computer memory, one or more floppy discs, compact discs (CD), optical discs, digital video disks (DVD), magnetic tapes, flash memories, RAM, ROM, EEPROM, circuit configurations in Field Programmable Gate Arrays or other semiconductor devices, or other tangible computer storage medium) encoded with one or more programs that, when executed on one or more computers or other processors, perform methods that implement the various embodiments discussed above. As is apparent from the foregoing examples, a computer readable storage medium may retain information for a sufficient time to provide computer-executable instructions in a non-transitory form. Such a computer readable storage medium or media can be transportable, such that the program or programs stored thereon can be loaded onto one or more different computing devices or other processors to implement various aspects of the present disclosure as discussed above. As used herein, the term “computer-readable storage medium” encompasses only a non-transitory computer-readable medium that can be considered to be a manufacture (i.e., article of manufacture) or a machine. Alternatively or additionally, the disclosure may be embodied as a computer readable medium other than a computer-readable storage medium, such as a propagating signal.

The terms “program” or “software” are used herein in a generic sense to refer to any type of computer code or set of computer-executable instructions that can be employed to program a computing device or other processor to implement various aspects of the present disclosure as discussed above. Additionally, it should be appreciated that according to one aspect of this embodiment, one or more computer programs that when executed perform methods of the present disclosure need not reside on a single computing device or processor, but may be distributed in a modular fashion amongst a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Various aspects of the present disclosure may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

The embodiments described herein may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Further, some actions are described as taken by a “user.” It should be appreciated that a “user” need not be a single individual, and that in some embodiments, actions attributable to a “user” may be performed by a team of individuals and/or an individual in combination with computer-assisted tools or other mechanisms.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art. Accordingly, the foregoing description and drawings are by way of example only.

1. A method of preparing an implantable material comprising:
obtaining a target configuration for a biological valve;
obtaining one or more characteristics of the biological valve to be reconstructed;
obtaining one or more mechanical characteristics of the implantable material; and
determining, based at least in part on the target configuration, the one or more biological valve characteristics, and the one or more mechanical characteristics of the implantable material, a pattern for the implantable material configured to reconstruct the biological valve.

2. The method of claim 1, wherein obtaining a target configuration for the biological valve includes obtaining a target size and/or shape of the implantable material.

3. The method of claim 1, further including cutting the implantable material into the determined pattern.

4. The method of claim 1, wherein measuring the one or more characteristics of the biological valve to be reconstructed measuring the one or more characteristics of the biological valve in a resting configuration.

5. The method of claim 1, wherein measuring the one or more characteristics of the biological valve to be reconstructed includes constructing a three-dimensional model of the biological valve.

6. The method of claim 1, wherein measuring the one or more characteristics of the biological valve to be reconstructed includes obtaining a stress-strain relationship of the biological valve.

7. The method of claim 1, wherein the pattern is further configured to improve postoperative function of the biological valve based at least in part on a three-dimensional spatial geometry of the biological valve, mechanical characteristics of the biological valve, the mechanical characteristics of the implantable material, a size of the implantable material, a shape of the implantable material, and/or an orientation of the implantable material.

8. The method of claim 1, wherein obtaining the one or more mechanical characteristics of the implantable material includes obtaining a stress-strain relationship of the implantable material.

9. The method of claim 8, wherein obtaining the stress-strain relationship of the implantable material includes relating a force per unit cross-section of the implantable material to a measure of an average deformation normalized to a size of the implantable material.

10. The method of claim 8, wherein obtaining the stress-strain relationship of the implantable material includes obtaining stress-strain measurements of the implantable material in two or more different directions.

11. The method of claim 1, wherein the implantable material is anisotropic.

12. The method of claim 6, wherein obtaining the stress-strain relationship of the biological valve includes relating a force per unit cross-section of the biological valve to a measure of an average deformation normalized to a size of the biological valve.

13. The method of claim 6, wherein obtaining the stress-strain relationship of the biological valve includes obtaining stress-strain measurements of the biological valve in two or more different directions.

14. The method of claim 1, wherein the biological valve is anisotropic.

15. The method of claim 1, further including projecting the determined pattern onto the implantable material.

16. The method of claim 1, wherein the biological valve is configured to separate a higher pressure biological region from a lower pressure biological region.

17. A patterned implantable material made according to the method of claim 1.

18. The method of claim 1, further including obtaining one or more characteristics of patient growth, wherein determining the pattern is further based at least in part on the characteristics of patient growth.

19. The method of claim 18, wherein determining the pattern based at least in part on the characteristics of patient growth includes oversizing the pattern.

20. A non-transitory computer readable storage media-medium comprising processor executable instructions that when executed perform a method for preparing an implantable material comprising the steps of:
obtaining a target configuration for a biological valve;
obtaining one or more characteristics of the biological valve to be reconstructed;
obtaining one or more mechanical characteristics of the implantable material; and
determining, based at least in part on the target configuration, the one or more biological valve characteristics, and the one or more mechanical characteristics of the implantable material, a pattern for the implantable material configured to reconstruct the biological valve.

21. The non-transitory computer readable storage medium of claim 20, wherein obtaining a target configuration for the biological valve includes obtaining a target size and/or shape of the implantable material.

22. The non-transitory computer readable storage medium of claim 20, further including cutting the implantable material into the determined pattern.

23. The non-transitory computer readable storage medium of claim 20, wherein measuring the one or more characteristics of the biological valve to be reconstructed includes measuring the one or more characteristics of the biological valve in a resting configuration.

24. The non-transitory computer readable storage medium of claim 20, wherein measuring the one or more characteristics of the biological valve to be reconstructed includes constructing a three-dimensional model of the biological valve.

25. The non-transitory computer readable storage medium of claim 20, wherein measuring the one or more characteristics of the biological valve to be reconstructed includes obtaining a stress-strain relationship of the biological valve.

26. The non-transitory computer readable storage medium of claim 20, wherein the pattern is further configured to improve postoperative function of the biological valve based at least in part on a three-dimensional spatial geometry of the biological valve, mechanical characteristics of the biological valve; the mechanical characteristics of the implantable material, a size of the implantable material, a shape of the implantable material, and/or an orientation of the implantable material.

27. The non-transitory computer readable storage medium of claim 20, wherein obtaining the one or more mechanical characteristics of the implantable material includes obtaining a stress-strain relationship of the implantable material.

28. The non-transitory computer readable storage medium of claim 27, wherein obtaining the stress-strain relationship of the implantable material includes relating a force per unit cross-section of the implantable material to a measure of an average deformation normalized to a size of the implantable material.

29. The non-transitory computer readable storage medium of claim 27, wherein obtaining the stress-strain relationship of the implantable material includes obtaining stress-strain measurements of the implantable material in two or more different directions.

30. The non-transitory computer readable storage medium of claim 20, wherein the implantable material is anisotropic.

31. The non-transitory computer readable storage medium of claim 23, wherein obtaining the stress-strain relationship of the biological valve includes relating a force per unit cross-section of the biological valve to a measure of an average deformation normalized to a size of the biological valve.

32. The non-transitory computer readable storage medium of claim 25, wherein obtaining the stress-strain relationship of the biological valve includes obtaining stress-strain measurements of the biological valve in two or more different directions.

33. The non-transitory computer readable storage medium of claim 20, wherein the biological valve is anisotropic.

34. The non-transitory computer readable storage medium of claim 20, further including projecting the determined pattern onto the implantable material.

35. The non-transitory computer readable storage medium of claim 20, wherein the biological valve is configured to separate a higher pressure biological region from a lower pressure biological region.

36. A patterned implantable material made using the non-transitory computer readable storage medium of claim 20.

37. The non-transitory computer readable storage medium of claim 20, further including obtaining one or more characteristics of patient growth, wherein determining the pattern is further based at least in part on the characteristics of patient growth.

38. The non-transitory computer readable storage medium of claim 37, wherein determining the pattern based at least in part on the characteristics of patient growth includes oversizing the pattern.
